(19) United States
(12) Patent Application Publication
(10) Pub. No.: US 2005/0100577 A1
(43) Pub. Date: May 12, 2005

(54) Expandable medical device with beneficial agent matrix formed by a multi solvent system

(76) Inventors: Theodore L. Parker, Danville, CA (US); John F. Shanley, Redwood City, CA (US); Micheline Lisa Markey, Santa Cruz, CA (US)

Correspondence Address: CINDY A. LYNCH;CONOR MEDSYSTEMS, INC., 1003 HAMILTON COURT, MENLO PARK, CA, 94025, US

(21) Appl. No.: 10/830,566

(22) Filed: Apr. 22, 2004

Related U.S. Application Data
(63) Continuation-in-part of application No. 10/705,151, filed on Nov. 10, 2003.

(51) Int. Cl. A61K 38/28; A61K 31/7076; A61K 31/445; A61F 2/00; A61K 9/22

(52) U.S. Cl. 424/423; 604/890.1; 514/3; 514/46

(57) ABSTRACT

A multi solvent drug delivery matrix formation method is used to place layers into a reservoir in a stent in a stepwise manner to achieve extended delivery of water soluble, sensitive, or difficult to deliver drugs. The multi solvent matrix formation method allows the formation of a drug reservoir with a layered morphology in which the mixing between layers is limited to allow the different layers to perform different functions in controlling drug delivery. A stent having a drug delivery matrix includes a first beneficial agent layer affixed to the stent by depositing a first solution of a first polymer and a first solvent, and a second beneficial agent layer affixed to the first beneficial agent layer by depositing a second solution of a second polymer and a second solvent. The second solvent is selected so that the first polymer is substantially insoluble in the second solvent to prevent degradation of the first polymer during deposition of the second polymer. A therapeutic agent is provided in the first beneficial agent layer or the second beneficial agent layer to form a drug delivery matrix.

### CROSS REFERENCE TO RELATED APPLICATIONS

This application is a Continuation-In-Part of U.S. patent application Ser. No. 10/705,151, filed on Nov. 10, 2003, which is incorporated herein by reference in its entirety.

### BACKGROUND

Implantable medical devices are often used for delivery of a therapeutic agent, such as a drug, to an organ or tissue in the body at a controlled delivery rate over an extended period of time. These devices may be able to be used to deliver agents to a wide variety of bodily systems to provide a variety of treatments.

One of the implantable medical devices which have been used for local delivery of therapeutic agents is the stent. Stents are typically introduced percutaneously, and transported transluminally until positioned at a desired location within a body lumen. These devices are then expanded either mechanically, such as by the expansion of a mandrel or balloon positioned inside the device, or expand themselves by releasing stored energy upon actuation within the body. Once expanded within a body lumen, the stent becomes encapsulated within the body tissue and remains a permanent implant.

Of the many problems that may be addressed through stent-based local delivery of therapeutic agents, one of the most important is restenosis. Restenosis is a major complication that can arise following vascular interventions such as angioplasty and the implantation of stents. Simply defined, restenosis is a wound healing process that reduces the vessel lumen diameter by extracellular matrix deposition, neointimal hyperplasia, and vascular smooth muscle cell proliferation, and which may ultimately result in renarrowing or even reocclusion of the vessel lumen. Despite the introduction of improved surgical techniques, devices, and pharmaceutical agents, the overall restenosis rate is still reported in the range of 25% to 50% within six to twelve months after an angioplasty procedure. To treat this condition, additional revascularization procedures are frequently required, thereby increasing trauma and risk to the patient.

One of the techniques under development to address the problem of restenosis is the use of surface coatings of various therapeutic agents on stents. U.S. Pat. No. 5,716,981, for example, discloses a stent that is surface-coated with a composition comprising a polymer carrier and paclitaxel (a well-known compound that is commonly used in the treatment of cancerous tumors). Known surface coatings, however, can provide little actual control over the release kinetics of therapeutic agents. These coatings are generally very thin, typically 5 to 8 microns deep. The surface area of the stent, by comparison is very large, so that the entire volume of the therapeutic agent has a very short diffusion path to discharge into the surrounding tissue.

In addition, it is not currently possible to deliver some drugs with a surface coating for a variety of reasons. In some cases, the drugs are sensitive to water, other compounds, or conditions in the body which degrade the drugs. For example, some drugs lose substantially all their activity when exposed to water for a period of time. When the desired treatment time is substantially longer than the half life of the drug in water the drug cannot be delivered by know coatings. Other drugs, such as protein or peptide based therapeutic agents, lose activity when exposed to enzymes, pH changes, or other environmental conditions. And finally drugs that are soluble in water tend to be released from the coatings at an undesirably high rate and do not remain localized for a therapeutically useful amount of time. These types of drugs which are sensitive to compounds or conditions in the body often cannot be delivered using surface coatings.

One of the reasons that water soluble drugs are not retained by polymer matrices is due to a phenomenon called blooming. Blooming occurs when a solution containing matrix material, drug, and solvent is deposited. During evaporation of the solvent the drug tends to migrate to the surface of the matrix following the evaporating solvent. This results in a high concentration of drug at or near the evaporative surface. The drug near the surface is quickly eluted when it enters the high fluid environment of the body. Thus, blooming leads to quick release and a large initial burst of drug. Water soluble drugs are more vulnerable to the high bursts caused by blooming because water soluble drugs are quickly transmitted to bodily fluid.

Accordingly, it would be desirable to provide a medical device with a beneficial agent matrix morphology which modulates the release of the beneficial agent to achieve a programmed administration period and release rate.

### SUMMARY OF THE INVENTION

The present invention relates to an implantable medical device having a plurality of beneficial agent layers formed by a multi solvent formation method which substantially reduces mixing between layers creating a plurality of independent layers.

In accordance with one aspect of the invention, an implantable medical device includes an implantable device body having a plurality of openings, at least one base layer contained in the plurality of openings comprising a first polymer material that is soluble in a first solvent, and at least one therapeutic layer contained in the plurality of openings. The therapeutic layer comprising a first therapeutic agent and a second polymer material both of which are soluble in a common second solvent in which the first polymer material is substantially insoluble.

In accordance with another aspect of the invention, an implantable medical device includes an implantable device body having a plurality of openings, at least one therapeutic agent layer contained in the plurality of openings, wherein the therapeutic agent layer is formed with a first polymer, a first solvent, and a first therapeutic agent, and at least one cap layer contained in the plurality of openings adjacent the at least one therapeutic agent layer, the at least one cap layer is formed with a second polymer and a second solvent, wherein the first therapeutic agent is at most marginally soluble in the second solvent.

In accordance with an additional aspect of the invention, a method of loading an implantable medical device with a controlled release polymer drug matrix deposits a first solution of a first polymer, a first therapeutic agent, and a first solvent in which the first polymer and the first therapeutic agent are both soluble, evaporates the first solvent, deposits a second solution of a second polymer, and a second solvent in which the second polymer is soluble, wherein the first therapeutic agent is substantially insoluble in the second solvent, and evaporates the second solvent.

In accordance with a further aspect of the invention, a method of loading an implantable medical device with a controlled release polymer drug matrix deposits a first solution of a first polymer and a first solvent in which the first polymer is soluble, evaporates the first solvent, deposits a second solution of a second polymer, a first therapeutic agent, and a second solvent in which the second polymer and the first therapeutic agent are both soluble, wherein the first polymer is substantially insoluble in the second solvent, and evaporates the second solvent.

In accordance with yet a further aspect of the invention, a method of loading an implantable medical device with a controlled release polymer drug matrix in a plurality of layers creates a first layer by delivering a first solution of a first polymer and a first solvent and evaporating the first solvent, creates a second layer by delivering a second solution of a second polymer and a second solvent, wherein the second solvent does not significantly dissolve the first layer, and provides a therapeutic agent in at least one of the first and second layers.

In accordance with yet a further aspect of the invention, an implantable stent includes an expandable stent body, a first beneficial agent layer affixed to the stent by depositing a first solution comprising a first polymer and a first solvent, wherein the first polymer is soluble in the first solvent, a second beneficial agent layer affixed to the first beneficial agent layer by depositing a second solution comprising a second polymer and a second solvent, wherein the second polymer is soluble in the second solvent and the first polymer is substantially insoluble in the second solvent, and a therapeutic agent provided in the first beneficial agent layer or the second beneficial agent layer, wherein the therapeutic agent is soluble in the first or second solvent.

In accordance with yet a further aspect of the invention, an implantable medical device comprises an implantable device body having a plurality of openings, at least two hydrophobic layers of matrix material in the openings, and at least one hydrophilic therapeutic agent layer in the openings positioned between the hydrophobic layers.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a perspective view of one example of a stent according to the present invention.

FIG. 2 is a side view of a portion of the stent of FIG. 1 which has been laid flat for ease of illustration.

FIG. 3 is a side cross sectional view of an example of a hole in a stent showing a base layer, a therapeutic agent layer, and a cap layer for extending release.

FIG. 4 is a side cross sectional view of an example of a hole in a stent showing a base layer, two therapeutic agent layers, and a cap layer for release of two therapeutic agents.

FIG. 5 is a side cross sectional view of an example of a hole in a stent showing two therapeutic agent layers separated by a separating layer for delivery from opposite sides of the stent.

FIG. 6 is a side cross sectional view of an example of a hole in a stent showing therapeutic agent layers separated by intermediate polymer layers for delayed agent delivery.

FIG. 7 is a graph of the cumulative release of insulin from stents formed as described in Example 1 with and without the dual solvent formation method.

FIG. 8 is a graph of the cumulative release of dA from stents formed as described in Example 2 with and without the dual solvent formation method.

### DETAILED DESCRIPTION

A multi solvent method is used to place layers into a reservoir in a stent in a stepwise manner to achieve controlled delivery of water soluble, sensitive, or difficult to deliver drugs. The multi solvent matrix formation method allows the formation of a drug reservoir with a layered morphology in which the mixing between layers is limited to allow the different layers to perform different functions in controlling drug delivery. The multi solvent matrix formation method employs different solvents for depositing different layers within the drug delivery matrix to substantially reduce mixing between the layers and to control the drug delivery.

Definitions

The following terms, as used herein, shall have the following meanings:

The term “beneficial agent” as used herein is intended to have the broadest possible interpretation and is used to include any therapeutic agent or drug, as well as inactive agents such a s barrier layers, carrier layers, therapeutic layers, separating layers, or protective layers.

The terms “drug” and “therapeutic agent” are used interchangeably to refer to any therapeutically active substance that is delivered to a living being to produce a desired, usually beneficial, effect.

The terms “matrix” or “biocompatible matrix” are used interchangeably to refer to a medium or material that, upon implantation in a subject, does not elicit a detrimental response sufficient to result in the rejection of the matrix. The matrix may contain or surround a therapeutic agent, and/or modulate the release of the therapeutic agent into the body. A matrix is also a medium that may simply provide support, structural integrity or structural barriers. The matrix may be polymeric, non-polymeric (e.g. carbohydrates and/or saccarides), hydrophobic, hydrophilic, lipophilic, amphiphilic, mixtures thereof and the like. The matrix may be bioresorbable or non-bioresorbable.

The term “bioresorbable” refers to a matrix, as defined herein, that can be broken down by either chemical or physical process, upon interaction with a physiological environment. The matrix can erode or dissolve. A bioresorbable matrix serves a temporary function in the body, such as drug delivery, and is then degraded or broken into components that are metabolizable or excretable, over a period of time from minutes to years, preferably less than one year, while maintaining any requisite structural integrity in that same time period.

The term “openings” includes holes, through openings, grooves, channels, recesses, and the like.

The term “polymer” refers to molecules formed from the chemical union of two or more repeating units, called monomers. Accordingly, included within the term “polymer” may be, for example, dimers, trimers and oligomers. The polymer may be synthetic, naturally-occurring or semisynthetic. In preferred form, the term “polymer” refers to molecules which typically have a Mw greater than about 3000 and preferably greater than about 10,000 and a Mw that is less than about 10 million, preferably less than about a million and more preferably less than about 200,000. Examples of polymers include but are not limited to, poly-α-hydroxy acid esters such as, polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); poly(vinylpyrrolidone) (PVP); polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclodextrin sulfobutyl ethers; polypeptides and proteins, such as polylysine, polyglutamic acid, albumin; polyanhydrides; polyhydroxy alkonoates such as polyhydroxy valerate, polyhydroxy butyrate, and the like.

The term “primarily” with respect to directional delivery, refers to an amount greater than about 50% of the total amount of therapeutic agent provided to a blood vessel.

The term “restenosis” refers to the renarrowing of an artery following an angioplasty procedure which may include stenosis following stent implantation.

The term “substantially linear release profile” refers to a release profile defined by a plot of the cumulative drug released versus the time during which the release takes place in which the linear least squares fit of such a release profile plot has a correlation coefficient value, r², of greater than 0.92 for data time points after the first day of delivery.

The term “water soluble drug” refers to drugs having a water solubility of about 0.1 mg/ml or greater.

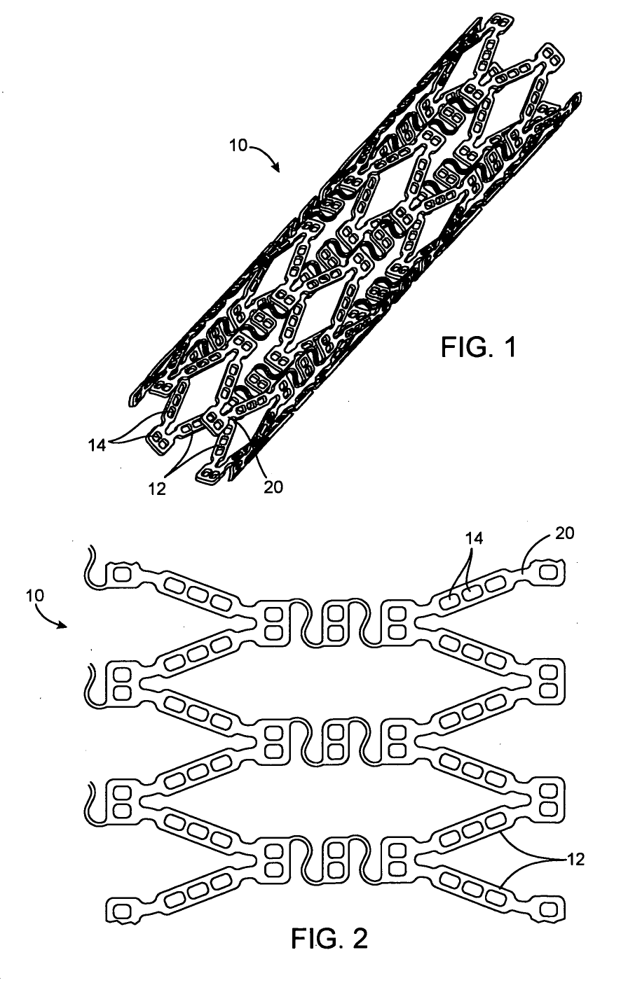

FIG. 1 illustrates one example of an implantable medical device in the form of a stent 10. FIG. 2 is an enlarged flattened view of a portion of the stent of FIG. 1 illustrating one example of a stent structure including struts 12 interconnected by ductile hinges 20. The struts 12 include openings 14 which can be non-deforming openings containing a therapeutic agent. One example of a stent structure having non-deforming openings is shown in U.S. Pat. No. 6,562,065 which is incorporated herein by reference in its entirety.

The implantable medical devices of the present invention are configured to release at least one therapeutic agent from a matrix affixed to the implantable body. The matrix is formed by a multi solvent formation method which allows the sequential assembly of a layered morphology to precisely control the rate of elution of the agent from the device.

A problem in loading water soluble or sensitive drugs in a reservoir in a stepwise manner when the same solvent is used for each layer is that as each layer is deposited, the underlying layer is partially dissolved by the solvent causing mixing of the drug throughout the matrix. When the drug is present throughout the reservoir matrix, the drug may be delivered almost immediately upon implantation or even during delivery of the stent. When blooming occurs the rapid release of drug is further accelerated. When water soluble drugs are delivered, the initial burst caused by blooming is accentuated. When an extended delivery period is desired, such as delivery over a period of about 1 day or more, the multi solvent matrix formation method of the invention provides a solution. The multi solvent method also effectively controls the initial burst.

A typical layered morphology formed with the multi solvent method includes a base layer which is the first layer to be delivered, a cap layer which is the last layer to be delivered, and a therapeutic agent layer there between. The therapeutic agent layer can include a therapeutic agent in combination with one or more matrix materials which serve the function of stabilizing the drug and maintaining bioactivity. The base and cap layers serve the function of modulating release rate and direction of release of the drug. The base and cap layers formed by the multi solvent system can contain substantially no therapeutic agent.

The multi solvent matrix formation method employs different solvents for depositing different layers within a drug delivery matrix to substantially reduce mixing between the layers and to control the drug delivery. The multi solvent method can be used to form a polymer inlay containing a water soluble or otherwise sensitive drug, such as 2-chlorodeoxyadenosine (2-CdA), insulin, other proteins, peptides, or water soluble small molecules, and one or more matrix layer without drug to achieve programmed delivery of the drug.

In one example of the multi solvent method as will be described in further detail below, a base layer and a cap layer can be formed by a material soluble in a different solvent from the therapeutic agent layers to prevent intermixing of these layers. In addition to the base layer and cap layer, other therapeutic agent layers, barrier layers, protective layers, or separating layers may also be formed of non-mixing combinations by selection of solvents in this manner.

In one embodiment, the matrix is a polymeric material which acts as a binder or carrier to hold the agent in or on the stent and/or modulate the release of the agent from the stent. The polymeric material can be a bioresorbable or a non-bioresorbable material. The matrix can also include a polymer in combination with one or more non-polymer matrix materials including carbohydrates and/or sacarides (sucrose, trehalose, mannitol). For example, a combination of PVP and sucrose.

The therapeutic agent containing matrix can be disposed in or on surfaces of the stent in various configurations, including within volumes defined by the stent, such as openings, holes, through holes, recesses, channels, or concave surfaces, as a reservoir of agent, or arranged in or on all or a portion of the surfaces of the stent structure. When the therapeutic agent matrix is disposed within openings in the strut structure of the stent to form a reservoir, the openings may be partially or completely filled with matrix containing the therapeutic agent.

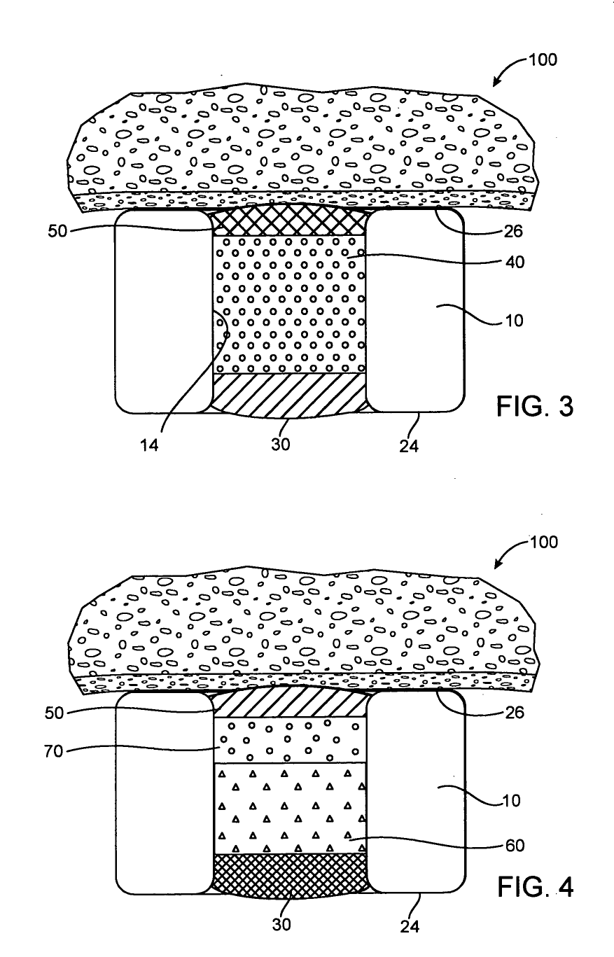

FIG. 3 is a cross section of the stent 10 illustrating one example of a strut opening 14 arranged adjacent a vessel wall 100 with a mural surface 26 of the stent abutting the vessel wall and a luminal surface 24 opposite the mural surface. The opening 14 of FIG. 3 contains a therapeutic agent layer 40 which includes a therapeutic agent in a biocompatible matrix, such as a bioresorbable polymer matrix. The therapeutic agent is illustrated by Os in the matrix. The luminal side 24 of the stent opening 14 is provided with a base layer 30. The opening is also provided with a cap layer 50 at the mural side. The base layer 30 and cap layer 50 control the direction of release and the release rate. On of the base and cap layers 30, 50 can serve as a barrier layer substantially preventing delivery of the drug to a particular side of the stent. The barrier layer erodes more slowly than the therapeutic agent layer 40 containing the therapeutic agent and thus, causes the therapeutic agent to be delivered primarily to the opposite of the stent. Alternately, the barrier layer can be non-biodegradable.

In the example of FIG. 3, the base layer 30 controls delivery of the therapeutic agent into the vessel lumen. The base layer 30 also prevents or retards release of the therapeutic during delivery of the stent to the vessel. The base layer 30 can be erodible at the same rate or more quickly than the therapeutic agent layer 40. Alternately, the base layer 30 can be non-biodegradable or a slowly degrading material and can form a molecular diffusion barrier through which the therapeutic agent passes. The cap layer 50 in the example of FIG. 3 is a slowly eroding biocompatible material or a non-biodegradable material which functions as a barrier layer.

The base layer 30, the therapeutic agent layer 40, and the cap layer 50 are prevented from mixing substantially during formation by the use of the multi solvent method, wherein the solvent used for deposition of each of these layers 30, 40, 50 is selected so that it does not appreciably dissolve the components of the layer below including the matrix material or polymer, any therapeutic agents, and any additives. The arrangement of layers shown in FIG. 3 is useful for delivery of a single drug luminally, such as the insulin example described below in Example 1.

The multi solvent system allows layers of polymer with or without drug to be formed without substantial mixing of the layers. The ability to substantially reduce or prevent mixing of the layers allows the layers to serve different functions, such a providing directional delivery, controlling delivery, or delaying delivery. The ability to provide the layers with specific functions is particularly useful when delivering sensitive or water soluble drugs in treatments which require controlled or extended drug delivery.

Due to the high water content of the environment within the body in which stents and other drug delivery devices are implanted, drugs with relatively low water solubilites can still be released very quickly. It is difficult to deliver highly water soluble drugs and is also difficult to deliver many drugs which are considered only slightly or marginally water soluble. For example, it is difficult to extend the delivery period for water soluble drugs, such as 2-CdA (water solubility about 4.5 mg/ml), arginine (water solubility about 14 g/ml), insulin (water solubility about 20 mg/ml). These drugs when incorporated in an implantable device in a polymer matrix will tend to be dissolved quickly in the surrounding fluid environment of the body due to a combination of the high fluid environment of the body and a relatively small amount of drug to be delivered. Water soluble drugs as discussed herein include drugs having a water solubility of about 0.1 mg/ml or greater.

The use of a layered structure including a lipophilic/hydrophobic base layer 30 and a lipophilic/hydrophobic cap layer 50 on opposite sides of a hydrophilic therapeutic agent layer 40 can further control delivery of water soluble or sensitive drugs. The hydrophobic layers can be used to retard resorbtion of the therapeutic agent. The hydrophobic layers can each have a total thickness of about 10% to about 30% of the total thickness of the matrix structure. The therapeutic agent layer can have a thickness larger than the hydrophobic layers to accommodate a large amount of drug between hydrophobic layers. For example, the therapeutic agent layer can have a total thickness of about 30% to about 80% of the total thickness of the matrix structure. In one example, the base and cap layers have thicknesses of about 5 to about 50 μm, preferably about 15 to about 45 μm, and the therapeutic agent layer has a thickness of about 20 to about 150 μm, preferably about 20 to about 100 μm.

Some of the water soluble drugs which can be used in the present invention include insulin, proteins, peptides, arginine, and 2-CdA. The water soluble and sensitive drugs can be included in a stent in a dosage sufficient to reduce restenosis, to reduce tissue damage after myocardial infarction, to promote angiogenesis, to reduce thrombogenicity, or to stabilize vulnerable plaque. The water soluble drugs can also be provided in other types of implants to treat cancer, to promote angiogenesis, or to deliver other locally administered drugs within the body.

FIG. 4 illustrates an alternative embodiment of a stent 10 having an opening containing two therapeutic agents. According to FIG. 4, a base layer 30 is provided at a luminal side 24 of the stent 10, followed by a first therapeutic agent layer 60, a second therapeutic agent layer 70, and a cap layer 50. In this arrangement, one or more of the layers may be formed using a solvent which does not substantially erode the layer below to protect one or more sensitive or water soluble drugs within the layered drug and polymer inlay. In this example, up to four different solvents can be used for the four different layers. Alternately one of the solvents used in the lower two layers 30, 60 can be repeated in the later layers or another arrangement of repeating solvents can be used. Although the two therapeutic agents have been illustrated in different layers they may also be formed in the same layer either 1) from a solution containing both drugs or 2) from two different drug solutions deposited in layers which become mixed.

One example of a sensitive water soluble drug which is difficult to deliver is 2-chlorodeoxyadenosine (2-CdA), also called cladribine. 2-CdA can be delivered over an extended period by formation of a drug delivery device by the multi solvent method, such as the device of FIG. 4. Without the multi solvent method or another protection system, 2-CdA would be delivered with a large burst occurring almost immediately upon implantation or even during implantation of the stent. With the multi solvent method the administration period for delivery of 2-CdA can be extended to several hours, 24 hours, 10 days, or 30 days.

2-CdA is very soluble in a first solvent, DMSO, and has marginal solubility in a second solvent, anisole. Thus, the use of the second solvent, anisole, to form layers on the top of the 2-CdA layers prevents the 2-CdA from migrating to the top of the inlay in an effect referred to as blooming. A second drug, such as paclitaxel, which is soluble in anisole can be provided in the top layers. Therefore, the arrangement shown in FIG. 4 can include 2-CdA in the first therapeutic agent layer 60 and paclitaxel in the second therapeutic agent layer 70.

A base or barrier layer 30 can also be formed of one or more polymer layers to provide directional delivery of the 2-CdA to the mural side. The barrier layer can be formed of a polymer, such as PLLA, which is not soluble or only marginally soluble in the first solvent (DMSO). Thus, the addition of the 2-CdA layers will not appreciable redissolve the barrier layer and the 2-CdA will not be substantially distributed into the barrier layer.

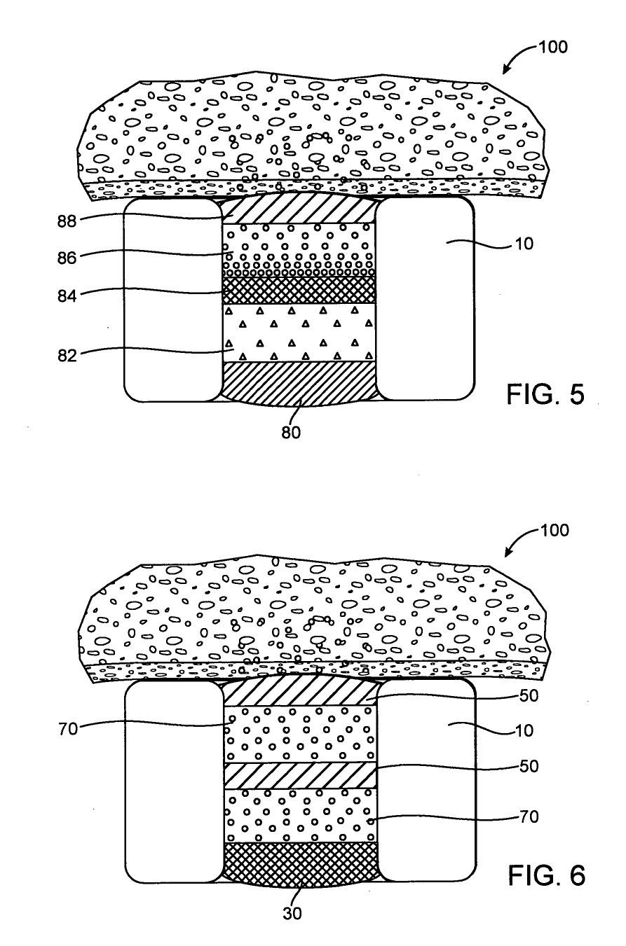

FIG. 5 illustrates another alternative embodiment of a stent 10 having an opening filled with two therapeutic agents arranged for dual direction delivery utilizing a multi solvent formation method. In FIG. 5, a first layer 80 is provided as a base layer on a luminal side of the stent, followed by a second layer 82 containing a first therapeutic agent represented by ▴s, a third layer 84 in the form of a separating or barrier layer, a fourth layer 86 containing a second therapeutic agent represented by Os, and a fifth layer 88 in the form of a cap layer at the mural side. The separating layer 84 can be formed of a slow degrading polymer material or non-biodegradable material which substantially prevents passage of the therapeutic agents through the separating layer. This arrangement can achieve directional delivery of the agent in the second layer 82 primarily luminally and delivery of the agent in the fourth layer 86 primarily murally. The separating layer 84 can be eliminated, for example where the two therapeutic agents are delivered over about the same administration period. To prevent or reduce mixing between layers as the layers are formed within the stent opening, one or more of the layers may be formed using a solvent which does not substantially erode the layer below to protect one or more sensitive or water soluble drugs within the layered drug and polymer inlay.

As can be seen in the example of FIG. 5, the concentration of the therapeutic agent (Os) in the therapeutic agent layer 86 is highest close to the barrier layer 84 of the stent 10 and lowest close to the cap layer 88. This configuration in which the drug can be precisely arranged within the matrix allows the release rate and administration period to be programmed to a particular application. The distribution of the therapeutic agent in the therapeutic agent layer 86 in addition to the use of the barrier layer 84 and the cap layer 88 together provide a programmable release rate and administration period. An arrangement such as the one shown in FIG. 5 can be used to achieve a substantially linear release rate of the second therapeutic agent from the stent 10 in the mural direction. Other arrangements of the therapeutic agent within the therapeutic agent layers can be used to produce other release profiles and/or release directions.

Generally, the therapeutic agent layers described herein are created in a plurality of steps of by delivery of a polymer/agent/solvent solution followed by drying and repeating. Since the therapeutic agent layers are formed in a plurality of independent steps which form a plurality of intermixed layers within the therapeutic agent layer, individual chemical compositions and pharmacokinetic properties can be imparted to each layer. Numerous useful arrangements of such layers within the therapeutic agent layer can be formed. Each of the layers may include one or more agents in the same or different proportions from layer to layer. Changes in the agent concentration between layers can be used to achieve a desired delivery profile. Substantially constant or linear release rates over time period from a few hours to months can be achieved.

Methods by which the drug can be precisely arranged within the matrix of the therapeutic agent layer in the openings by a stepwise deposition process is further described in U.S. patent application Ser. No. 10/777,283 filed on Feb. 11, 2004, which is incorporated herein by reference in its entirety.

The layers can be formed by a piezo-electric dispensing device which precisely deposits droplets into the openings. An example of such a device is described in U.S. patent application Ser. No. 10/668,125, filed on Sep. 22, 2003, which is incorporated herein by reference in its entirety.

FIG. 6 illustrates an alternative embodiment of a stent 10 having an opening filled with alternating therapeutic agent layers and polymer only layers to achieve a timed release of the therapeutic agent. In the FIG. 6 example, a base layer 30 on the luminal side of the opening serves as a barrier layer and is followed by alternating therapeutic agent layers 70 and separating/cap layers 50 without a significant amount of therapeutic agent. Although two drug layers 70 are shown, additional drug layers can also be used. Alternating drug layers 70 with separating or cap layers 50 provides extended and/or pulsatile delivery.

FIGS. 3-6 illustrates some of the many examples of the layered combinations which can be formed according to the present invention. Many other combinations and arrangements of layers can be used to deliver one or more agents, in one or more directions, with any number of release rates and administration periods for the different agents. The use of barrier layers (slow degrading or non-degrading layers), protective layers, separating layers, and cap layers together or individually can control the agent release rate and administration period of a therapeutic agent.

In one alternative embodiment, the barrier layer is a slow eroding layer which can be annealed (heat treating for an extended period) to remove substantially all the solvent from the base layer. Annealing as used herein means heating the base layer to a temperature higher then the Tg (glass transition temperature) but lower than the Tm (melting temperature). Annealing allows the polymer chains to move around and reposition themselves such that any possible channels which would allow drug to pass more quickly through the matrix are minimized or eliminated. This makes the base layer more impervious to a water soluble drug. Annealing removes additional solvent from the barrier layer and improves resistance of the barrier layer to subsequent erosion upon deposition of the therapeutic agent layers. Annealing results in a more compact crystalline structure of the material and slows the passage of drug through the base layer. In one example: A 3% PLLA in 10% TFE barrier layer was annealed at 100 deg C. for 30 minutes. Annealing can be used on each individual layer within the base layer or on the base layer as a whole. In some cases annealing can eliminating the need to use a different solvent in the base layer and therapeutic agent layer.

Wetting agents such as glycerol monostearate, calcium stearate, Poloxamer 407, sorbitan monostearate, vitamin E-TPGS, Lecithin, and the like can be used, particularly in the barrier layer or other first layer to improve the consistency of the first layer.

The solvents used for each layer can be selected to prevent dissolution of the layers below. The particular solvent properties can be adjusted to get a particular solubility profile by combining solvents in solvent blends. I many instances, the solvent is selected so that it does not dissolve either the polymer or the drug in the layer below. Alternatively, it may be desirable to allow one or more of the components to be partially dissolved by selecting the solvent properties.

The multi solvent process as described herein is used to form a plurality of layers deposited sequentially within an opening in a stent. The multi solvent system can be used with stents having different strut configurations including coil, woven, serpentine, diamond shaped, chevron shaped, or other strut configurations. The multi solvent system can also be used with other implantable medical devices, such as implantable drug delivery devices including coils, meshes, filaments, discs, cylinders, or other shaped drug delivery devices. Additionally, the multi solvent system can also be used to create layered polymer/drug matrices on the surfaces of or inside implantable medical devices. The volume of the openings in one example of the present invention is about 0.1 nanoliters to about 50 nanoliters.

The arrangement of the layers formed by the multi solvent process also controls the duration of release or administration period which may be a short release of 1-24 hours, moderate release of about 1 to about 7 days, or extended release of about 7 or more days. Each of the areas of the matrix may include one or more agents in the same or different proportions from one area to the next. The agents may be homogeneously disposed or heterogeneously disposed in different areas of the matrix.

The layers described herein may be solid, porous, or filled with other drugs or excipients. Although in the examples described herein, each of the layers is in a solid state when the drug delivery device is delivered to the body, one or more of the layers can also be in liquid or gel form when delivered. For example, a liquid or gel therapeutic agent layer can be arranged between solid barrier and cap layers. Alternately, a quick degrading layer, such as a cap layer, can be formed as a gel.

Although the present invention has been described as employing a base layer and a cap layer, one or the other of these layers may be omitted. For example, when the therapeutic agent matrix is deposited into a well, recess, or channel having a bottom, a base layer is not used. Alternately, a tapered opening having a narrow bottom can be used to control delivery without a base layer.

### EXAMPLE 1

### Preparation of Stents Containing Insulin

Stents of the general configuration illustrated in FIG. 1 were mounted on a mandrel and individual holes were filled with and without the multiple solvent system to show the reduction in burst achievable using the dual solvent system.

Fast Release—Soluble Base Layer

In the fast release example, a base layer was formed by multiple steps of filling with a solution of 5% poly(lactide-co-glycolide) (PLGA) in anisole, the solution was dried between filling steps. A drug layer was then formed in multiple steps of filling with a solution of 10% insulin, 10% poly(vinylpyrrolidone)(PVP) in DMSO, the solution was dried between filling steps. Since PLGA is soluble in DMSO, the DMSO will partially dissolve the PLGA. A cap layer was then formed in multiple steps of filling with a solution of 5% poly(lactide-co-caprolactone)(PLA-PCL) in anisole with the solution dried between filling steps. The PVP is not soluble in anisole and thus, the cap layer does not appreciably mix with the drug layer.

Slow Release—Insoluble Base Layer

In the slow release example, a base layer was formed by multiple steps of filling with a solution of 3% poly(L-lactide) (PLLA) in a solvent blend of one or more of anisole, trifluoroethanol, methylene chloride, hexafluoroisopropanol (HFIP), trifluoroethanol (TFE), heptafluorobutanol (HFB), and chloroform, the solution was dried between filling steps. A drug layer was then formed in multiple steps of filling with a solution of 10% insulin, 10% poly(vinylpyrrolidone)(PVP) in DMSO, the solution was dried between filling steps. Since PLLA is insoluble in DMSO, the DMSO will not appreciably dissolve the PLLA base layer. A cap layer was then formed in multiple steps of filling with a solution of 5% PLGA in anisole with the solution dried between filling steps. The PVP is not soluble in anisole and thus, the cap layer does not appreciably mix with the drug layer.

In each case, multiple steps of filling with each solution were used to achieve the desired thickness of a composition or amount of a drug. When all the filling steps were completed a total of about 200 micrograms of insulin had been placed into the reservoirs of a stent having a length of about 17 mm.

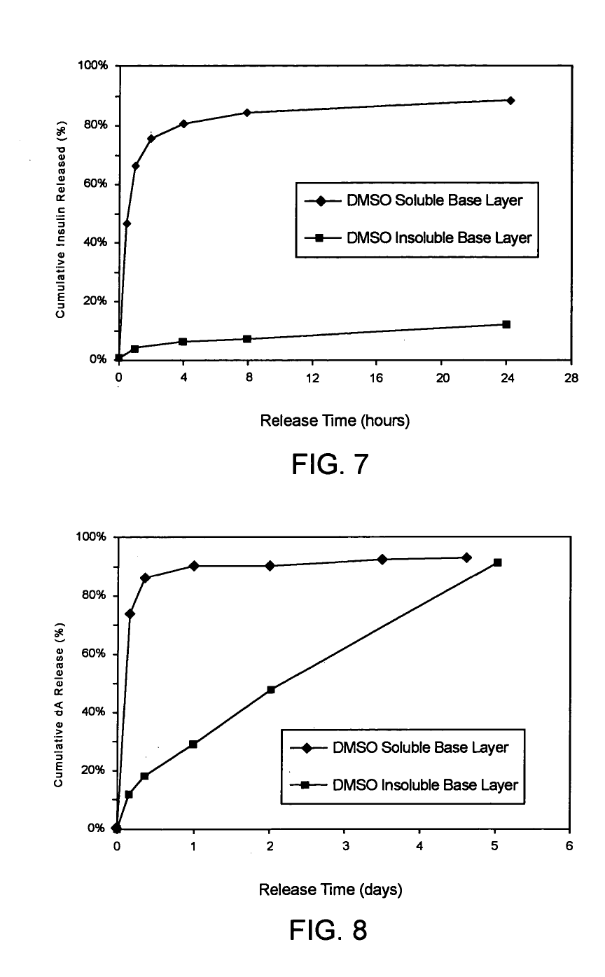

A series of plastic vials were charged with 1.0 ml of phosphate buffered saline (PBS) solution, then placed in a water bath held at 37 degrees C. and shaking at 20 cpm under so-called “infinite sink” conditions. A sample from the above prepared stent lot was placed in the first release vial, held for 4 hours, then removed and transferred to the next fresh release solution vial. The process was repeated to gather release samples over 24 hours. After the 24 hour data point, the stent was extracted into 1.0 ml DMSO solvent to gather any insulin residual on the stent. Each vial was assayed for insulin content by HPLC analysis. The cumulative percentage amount of insulin released for the fast and slow release formulations is shown in the graph of FIG. 7. As can be seen in the graph, the fast release with the base layer formed of a DMSO soluble material resulted in a initial burst of over 70% of the insulin within the first two hours. The slow release formulation with the base layer formed of a DMSO insoluble material results in a significant decrease in the burst and a much more controlled release of insulin over the first 24 hours.

### EXAMPLE 2

### Preparation of Stent Containing dA

Stents of the general configuration illustrated in FIG. 1 were mounted on a mandrel and individual holes were filled with and without the multiple solvent system to show the reduction in burst achievable using the dual solvent system. The deoxyadenosine (dA) used in these formulations is used as a surrogate for 2-CdA and provides results which are comparable with 2-CdA.

Fast Release—Soluble Base Layer

In the fast release example, a base layer was formed by multiple steps of filling with a solution of 4% poly(lactide-co-glycolide) (PLGA) in dimethyl sulfoxide (DMSO), the solution was dried between filling steps. A drug layer was then formed in multiple steps of filling with a solution of 22.5% dA, 7.5% poly(vinylpyrrolidone)(PVP) in DMSO, the solution was dried between filling steps. Since PLGA is soluble in DMSO, the DMSO will partially dissolve the PLGA. A cap layer was then formed in multiple steps of filling with a solution of 5% PLGA in anisole with the solution dried between filling steps. The PVP is not soluble in anisole and thus, the cap layer does not appreciably mix with the drug layer.

Slow Release—Insoluble Base Layer

In the slow release example, a base layer was formed by multiple steps of filling with a solution of 3% poly(L-lactide) (PLLA) in a solvent blend of one or more of anisole, trifluoroethanol, methylene chloride, hexafluoroisopropanol (HFIP), trifluoroethanol (TFE), heptafluorobutanol (HFB), and chloroform, the solution was dried between filling steps. A drug layer was then formed in multiple steps of filling with a solution of 10% dA, 10% poly(vinylpyrrolidone)(PVP) in DMSO, the solution was dried between filling steps. Since PLLA is insoluble in DMSO, the DMSO will not appreciably dissolve the PLLA base layer. A cap layer was then formed in multiple steps of filling with a solution of 5% PLGA in anisole with the solution dried between filling steps. The PVP is not soluble in anisole and thus, the cap layer does not appreciably mix with the drug layer.

In each case, multiple steps of filling with each solution were used to achieve the desired thickness of a composition or amount of a drug. When all the filling steps were completed a total of about 175 micrograms of dA had been placed into the reservoirs of a stent having a length of about 17 mm.

The cumulative percentage amount of dA released for the fast and slow release formulations was determined as in Example 1 and is shown in the graph of FIG. 8. As can be seen in the graph, the fast release with the base layer formed of a DMSO soluble material resulted in a initial burst of over 70% of the dA within the first four hours. The slow release formulation with the base layer formed of a DMSO insoluble material results in a significant decrease in the burst and a much more controlled release of the dA over five days.

Therapeutic Agents

Other therapeutic agents for use with the present invention which may be use alone or in combination may, for example, take the form of small molecules, peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense oligonucleotides, monoclonal antibodies, platelets, prions, viruses, bacteria, eukaryotic cells such as endothelial cells, stem cells, ACE inhibitors, monocyte/macrophages and vascular smooth muscle cells. Such agents can be used alone or in various combinations with one another. For instance, anti-inflammatories may be used in combination with antiproliferatives to mitigate the reaction of tissue to the antiproliferative. The therapeutic agent may also be a pro-drug, which metabolizes into the desired drug when administered to a host. In addition, therapeutic agents may be pre-formulated as microcapsules, micro spheres, micro bubbles, liposomes, niosomes, emulsions, dispersions or the like before they are incorporated into the matrix. Therapeutic agents may also be radioactive isotopes or agents activated by some other form of energy such as light or ultrasonic energy, or by other circulating molecules that can be systemically administered.

Exemplary classes of therapeutic agents include antiproliferatives, antithrombins (i.e., thrombolytics), immunosuppressants, antilipid agents, anti-inflammatory agents, antineoplastics including antimetabolites, antiplatelets, angiogenic agents, anti-angiogenic agents, vitamins, antimitotics, metalloproteinase inhibitors, NO donors, nitric oxide release stimulators, anti-sclerosing agents, vasoactive agents, endothelial growth factors, beta blockers, hormones, statins, insulin growth factors, antioxidants, membrane stabilizing agents, calcium antagonists (i.e., calcium channel antagonists), retinoids, anti-macrophage substances, antilymphocytes, cyclooxygenase inhibitors, immunomodulatory agents, angiotensin converting enzyme (ACE) inhibitors, anti-leukocytes, high-density lipoproteins (HDL) and derivatives, cell sensitizers to insulin, prostaglandins and derivatives, anti-TNF compounds, hypertension drugs, protein kinases, antisense oligonucleotides, cardio protectants, petidose inhibitors (increase blycolitic metabolism), endothelin receptor agonists, interleukin-6 antagonists, anti-restenotics, and other miscellaneous compounds.

Antiproliferatives include, without limitation, sirolimus, paclitaxel, actinomycin D, rapamycin, and cyclosporin.

Antithrombins include, without limitation, heparin, plasminogen, a2-antiplasmin, streptokinase, bivalirudin, and tissue plasminogen activator (t-PA).

Immunosuppressants include, without limitation, cyclosporine, rapamycin and tacrolimus (FK-506), sirolumus, everolimus, etoposide, and mitoxantrone.

Antilipid agents include, without limitation, HMG CoA reductase inhibitors, nicotinic acid, probucol, and fibric acid derivatives (e.g., clofibrate, gemfibrozil, gemfibrozil, fenofibrate, ciprofibrate, and bezafibrate).

Anti-inflammatory agents include, without limitation, salicylic acid derivatives (e.g., aspirin, insulin, sodium salicylate, choline magnesium trisalicylate, salsalate, dflunisal, salicylsalicylic acid, sulfasalazine, and olsalazine), para-amino phenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (e.g., ibuprofen, naproxen, flurbiprofen, ketoprofen, fenoprofen, and oxaprozin), anthranilic acids (e.g., mefenamic acid and meclofenamic acid), enolic acids (e.g., piroxicam, tenoxicam, phenylbutazone and oxyphenthatrazone), alkanones (e.g., nabumetone), glucocorticoids (e.g., dexamethaxone, prednisolone, and triamcinolone), pirfenidone, and tranilast.

Antineoplastics include, without limitation, nitrogen mustards (e.g., mechlorethamine, cyclophosphamide, ifosfamide, melphalan, and chlorambucil), methylnitrosoureas (e.g., streptozocin), 2-chloroethylnitrosoureas (e.g., carmustine, lomustine, semustine, and chlorozotocin), alkanesulfonic acids (e.g., busulfan), ethylenimines and methylmelamines (e.g., triethylenemelamine, thiotepa and altretamine), triazines (e.g., dacarbazine), folic acid analogs (e.g., methotrexate), pyrimidine analogs (5-fluorouracil, 5-fluorodeoxyuridine, 5-fluorodeoxyuridine monophosphate, cytosine arabinoside, 5-azacytidine, and 2′,2′-difluorodeoxycytidine), purine analogs (e.g., mercaptopurine, thioguanine, azathioprine, adenosine, pentostatin, cladribine, and erythrohydroxynonyladenine), antimitotic drugs (e.g., vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin), phenoxodiol, etoposide, and platinum coordination complexes (e.g., cisplatin and carboplatin).

Antiplatelets include, without limitation, insulin, dipyridamole, tirofiban, eptifibatide, abciximab, and ticlopidine.

Angiogenic agents include, without limitation, phospholipids, ceramides, cerebrosides, neutral lipids, triglycerides, diglycerides, monoglycerides lecithin, sphingosides, angiotensin fragments, nicotine, pyruvate thiolesters, glycerol-pyruvate esters, dihydoxyacetone-pyruvate esters and monobutyrin.

Anti-angiogenic agents include, without limitation, endostatin, angiostatin, fumagillin and ovalicin.

Vitamins include, without limitation, water-soluble vitamins (e.g., thiamin, nicotinic acid, pyridoxine, and ascorbic acid) and fat-soluble vitamins (e.g., retinal, retinoic acid, retinaldehyde, phytonadione, menaqinone, menadione, and alpha tocopherol).

Antimitotics include, without limitation, vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin.

Metalloproteinase inhibitors include, without limitation, TIMP-1, TIMP-2, TIMP-3, and SmaPI.

NO donors include, without limitation, L-arginine, amyl nitrite, glyceryl trinitrate, sodium nitroprusside, molsidomine, diazeniumdiolates, S-nitrosothiols, and mesoionic oxatriazole derivatives.

NO release stimulators include, without limitation, adenosine.

Anti-sclerosing agents include, without limitation, collagenases and halofuginone.

Vasoactive agents include, without limitation, nitric oxide, adenosine, nitroglycerine, sodium nitroprusside, hydralazine, phentolamine, methoxamine, metaraminol, ephedrine, trapadil, dipyridamole, vasoactive intestinal polypeptides (VIP), arginine, and vasopressin.

Endothelial growth factors include, without limitation, VEGF (Vascular Endothelial Growth Factor) including VEGF-121 and VEG-165, FGF (Fibroblast Growth Factor) including FGF-1 and FGF-2, HGF (Hepatocyte Growth Factor), and Ang1 (Angiopoietin 1).

Beta blockers include, without limitation, propranolol, nadolol, timolol, pindolol, labetalol, metoprolol, atenolol, esmolol, and acebutolol.

Hormones include, without limitation, progestin, insulin, the estrogens and estradiols (e.g., estradiol, estradiol valerate, estradiol cypionate, ethinyl estradiol, mestranol, quinestrol, estrond, estrone sulfate, and equilin).

Statins include, without limitation, mevastatin, lovastatin, simvastatin, pravastatin, atorvastatin, and fluvastatin.

Insulin growth factors include, without limitation, IGF-1 and IGF-2.

Antioxidants include, without limitation, vitamin A, carotenoids and vitamin E.

Membrane stabilizing agents include, without limitation, certain beta blockers such as propranolol, acebutolol, labetalol, oxprenolol, pindolol and alprenolol.

Calcium antagonists include, without limitation, amlodipine, bepridil, diltiazem, felodipine, isradipine, nicardipine, nifedipine, nimodipine and verapamil.

Retinoids include, without limitation, all-trans-retinol, all-trans-14-hydroxyretroretinol, all-trans-retinaldehyde, all-trans-retinoic acid, all-trans-3,4-didehydroretinoic acid, 9-cis-retinoic acid, 11-cis-retinal, 13-cis-retinal, and 13-cis-retinoic acid.

Anti-macrophage substances include, without limitation, NO donors.

Anti-leukocytes include, without limitation, 2-CdA, IL-1 inhibitors, anti-CD116/CD18 monoclonal antibodies, monoclonal antibodies to VCAM, monoclonal antibodies to ICAM, and zinc protoporphyrin.

Cyclooxygenase inhibitors include, without limitation, Cox-1 inhibitors and Cox-2 inhibitors (e.g., CELEBREX® and VIOXX®).

Immunomodulatory agents include, without limitation, immunosuppressants (see above) and immunostimulants (e.g., levamisole, isoprinosine, Interferon alpha, and Interleukin-2).

ACE inhibitors include, without limitation, benazepril, captopril, enalapril, fosinopril sodium, lisinopril, quinapril, ramipril, and spirapril.

Cell sensitizers to insulin include, without limitation, glitazones, P par agonists and metformin.

Antisense oligonucleotides include, without limitation, resten-NG.

Cardio protectants include, without limitation, VIP, pituitary adenylate cyclase-activating peptide (PACAP), apoA-I milano, amlodipine, nicorandil, cilostaxone, and thienopyridine.

Petidose inhibitors include, without limitation, omnipatrilat.

Anti-restenotics include, without limitation, include vincristine, vinblastine, actinomycin, epothilone, paclitaxel, and paclitaxel derivatives (e.g., docetaxel).

Miscellaneous compounds include, without limitation, Adiponectin.

While the invention has been described in detail with reference to the preferred embodiments thereof, it will be apparent to one skilled in the art that various changes and modifications can be made and equivalents employed, without departing from the present invention.

1. An implantable medical device comprising:
an implantable device body having a plurality of openings;
at least one base layer contained in the plurality of openings comprising a first polymer material that is soluble in a first solvent; and
at least one therapeutic layer contained in the plurality of openings comprising a first therapeutic agent and a second polymer material both of which are soluble in a common second solvent in which the first polymer material is substantially insoluble.

2. The device of claim 1, wherein the at least one therapeutic agent is a water soluble drug.

3. The device of claim 2, wherein the at least one therapeutic agent is 2-chlorodeoxyadenosine.

4. The device of claim 2, wherein the at least one therapeutic agent is insulin.

5. The device of claim 1, wherein the at least one therapeutic agent and the second polymer material are soluble in dimethyl sulfoxide and the first polymer material is soluble in one or more of anisole, trifluoroethanol, methylene chloride, hexafluoroisopropanol (HFIP), trifluoroethanol (TFE), heptafluorobutanol (HFB), and chloroform, or mixtures thereof.

6. The device of claim 1, further comprising a cap layer formed of a third polymer material that is soluble in a third solvent in which the at least one therapeutic agent is substantially insoluble.

7. The device of claim 6, wherein the cap layer comprises a second therapeutic agent which is soluble in the third solvent.

8. The device of claim 7, wherein the second therapeutic agent is paclitaxel.

9. The device of claim 6, wherein the first and third solvents are the same.

10. The device of claim 1, wherein the first, second, and third polymer materials are bioerodible polymers.

11. The device of claim 10, wherein the first polymer material or the third polymer material is a slower degrading polymer than the second polymer material to provide directional delivery of the at least one therapeutic agent.

12. The device of claim 1, wherein the base layer is annealed to resist dissolution.

13. The device of claim 1, wherein the implantable medical device is a stent.

14. The device of claim 13, wherein the plurality of openings are radially oriented non-deforming through holes.

15. The device of claim 1, wherein the plurality of openings each have a volume of about 0.1 nanoliters to about 50 nanoliters.

16. The device of claim 1, wherein the at least one base layer does not include a substantial amount of the at least one therapeutic agent.

17. The device of claim 1, wherein the at least one therapeutic agent is arranged to be delivered over an administration period of about 7 days or more.

18. An implantable medical device comprising:
an implantable device body having a plurality of openings;
at least one therapeutic agent layer contained in the plurality of openings, wherein the therapeutic agent layer is formed with a first polymer, a first solvent, and a first therapeutic agent; and
at least one cap layer contained in the plurality of openings adjacent the at least one therapeutic agent layer, the at least one cap layer is formed with a second polymer and a second solvent, wherein the first therapeutic agent is at most marginally soluble in the second solvent.

19. The device of claim 18, wherein the at least one therapeutic agent is a water soluble drug.

20. The device of claim 19, wherein the at least one therapeutic agent is 2-chlorodeoxyadenosine.

21. The device of claim 19, wherein the at least one therapeutic agent is insulin.

22. The device of claim 18, wherein the cap layer includes a second therapeutic agent which is soluble in the second solvent.

23. The device of claim 22, wherein the second therapeutic agent is paclitaxel.

24. The device of claim 18, further comprising at least one base layer contained in the plurality of holes adjacent the at least one therapeutic agent layer, wherein the base layer is formed with a third polymer and a third solvent.

25. The device of claim 24, wherein the second and third polymers solvents are the same.

26. The device of claim 24, wherein the first, second, and third polymers are bioerodible polymers.

27. The device of claim 18, wherein the implantable medical device is a stent.

28. The device of claim 27, wherein the plurality of holes are radially oriented non-deforming through holes.

29. The device of claim 18, wherein the plurality of openings each have a volume of about 0.1 nanoliters to about 50 nanoliters.

30. The device of claim 18, wherein the at least one therapeutic agent is arranged to be delivered over an administration period of about 7 days or more.

31. The device of claim 18, wherein the at least one cap layer does not include a substantial amount of the at least one therapeutic agent.

32. A method of loading an implantable medical device with a controlled release polymer drug matrix:
depositing a first solution of a first polymer, a first therapeutic agent, and a first solvent in which the first polymer and the first therapeutic agent are both soluble;
evaporating the first solvent;
depositing a second solution of a second polymer, and a second solvent in which the second polymer is soluble, wherein the first therapeutic agent is substantially insoluble in the second solvent; and
evaporating the second solvent.

33. The method of claim 32, wherein the first solution and the second solution are deposited in a plurality of openings in the medical device.

34. The method of claim 33, wherein the plurality of openings are radially oriented non-deforming through holes.

35. The method of claim 32, wherein the first therapeutic agent is a water soluble drug.

36. The method of claim 35, wherein the first therapeutic agent is 2-chlorodeoxyadenosine.

37. The method of claim 35, wherein the first therapeutic agent is insulin.

38. The method of claim 32, wherein the second solution includes a second therapeutic agent which is soluble in the second solvent.

39. The method of claim 38, wherein the second therapeutic agent is paclitaxel.

40. The method of claim 32, wherein the first and second polymer materials are bioerodible polymers.

41. The method of claim 32, wherein the implantable medical device is a stent.

42. A method of loading an implantable medical device with a controlled release polymer drug matrix:
depositing a first solution of a first polymer and a first solvent in which the first polymer is soluble;
evaporating the first solvent;
depositing a second solution of a second polymer, a first therapeutic agent, and a second solvent in which the second polymer and the first therapeutic agent are both soluble, wherein the first polymer is substantially insoluble in the second solvent; and
evaporating the second solvent.

43. The method of claim 42, wherein the first solution and the second solution are deposited in a plurality of openings in the medical device.

44. The method of claim 43, wherein the plurality of openings are radially oriented non-deforming through holes.

45. The method of claim 42, wherein the first therapeutic agent is a water soluble drug.

46. The method of claim 42, wherein the first therapeutic agent is 2-chlorodeoxyadenosine.

47. The method of claim 42, wherein the first therapeutic agent is insulin.

48. The method of claim 42, wherein the first solution includes a second therapeutic agent which is soluble in the first solvent.

49. The method of claim 42, wherein the second therapeutic agent is paclitaxel.

50. The method of claim 42, wherein the first and second polymer materials are bioerodible polymers.

51. The method of claim 42, wherein the implantable medical device is a stent.

52. The method of claim 42, wherein the steps of depositing the first solution and evaporating the first solvent form at least one base layer and the subsequent deposition of the second solution results in an insubstantial amount of the first therapeutic agent in the base layer.

53. A method of loading an implantable medical device with a controlled release polymer drug matrix in a plurality of layers:
creating a first layer by delivering a first solution of a first polymer and a first solvent and evaporating the first solvent;
creating a second layer by delivering a second solution of a second polymer and a second solvent, wherein the second solvent does not significantly dissolve the first layer; and
providing a therapeutic agent in at least one of the first and second layers.

54. The method of claim 53, wherein the first polymer is selected from the group consisting of poly-L-lactide and PLGA and the second polymer is poly(vinylpyrrolidone).

55. The method of claim 53, wherein the first layer and the second layer are deposited in a plurality of openings in the medical device.

56. The method of claim 55, wherein the plurality of openings are radially oriented non-deforming through holes.

57. The method of claim 53, wherein the therapeutic agent is a water soluble drug.

58. The method of claim 57, wherein the therapeutic agent is 2-chlorodeoxyadenosine.

59. The method of claim 57, wherein the therapeutic agent is insulin.

60. The method of claim 53, wherein the first and second polymer materials are bioerodible polymers.

61. The method of claim 53, wherein the implantable medical device is a stent.

62. An implantable stent comprising:
an expandable stent body;
a first beneficial agent layer affixed to the stent by depositing a first solution comprising a first polymer and a first solvent, wherein the first polymer is soluble in the first solvent;
a second beneficial agent layer affixed to the first beneficial agent layer by depositing a second solution comprising a second polymer and a second solvent, wherein the second polymer is soluble in the second solvent and the first polymer is substantially insoluble in the second solvent; and
a therapeutic agent provided in the first beneficial agent layer or the second beneficial agent layer, wherein the therapeutic agent is soluble in the first or second solvent.

63. The stent of claim 62, wherein the first and second beneficial agent layers are deposited in a plurality of openings in the stent body.

64. The stent of claim 63, wherein the plurality openings are radially oriented non-deforming through holes.

65. The stent of claim 62, wherein the therapeutic agent is a water soluble drug.

66. The stent of claim 65, wherein the therapeutic agent is 2-chlorodeoxyadenosine.

67. The stent of claim 65, wherein the therapeutic agent is insulin.

68. The stent of claim 62, wherein the therapeutic agent is soluble in the first solvent and is substantially insoluble in the second solvent.

69. The stent of claim 62, wherein the therapeutic agent is soluble in the second solvent and is substantially insoluble in the first solvent 70. The device of claim 63, wherein the plurality of openings each have a volume of about 0.1 nanoliters to about 50 nanoliters.

70. The device of claim 65, wherein the therapeutic agent is arranged to be delivered from the stent over an administration period of about 7 days or more.

71. An implantable medical device comprising:
an implantable device body having a plurality of openings;
at least two hydrophobic layers of matrix material in the openings; and
at least one hydrophilic therapeutic agent layer in the openings positioned between the hydrophobic layers.

72. A method of loading an implantable medical device with a controlled release polymer drug matrix in a plurality of layers:
creating a base layer by delivering a first solution of a first polymer and a first solvent and evaporating the first solvent;
annealing the base layer;
creating a therapeutic agent layer by delivering a second solution of a second polymer, a therapeutic agent, and a second solvent.

73. The method of claim 72, wherein the base layer contains substantially no therapeutic agent.

74. The method of claim 72, wherein the steps of delivering the first solution, evaporating the first solvent, and annealing are repeated for multiple layers of the base layer.
